(19) United States
(12) Patent Application Publication
(10) Pub. No.: US 2007/0001023 A1
(43) Pub. Date: Jan. 4, 2007

(54) Air freshener

(76) Inventors: Dennis E. Green, Englewood, CO (US); Mary Lou Green, Englewood, CO (US)

Correspondence Address: DORR, CARSON & BIRNEY, P.C.;ONE CHERRY CENTER, 501 SOUTH CHERRY STREET, SUITE 800, DENVER, CO, 80246, US

(21) Appl. No.: 11/156,799

(22) Filed: Jun. 20, 2005

(51) Int. Cl. A24F 25/00; A61L 9/04

(52) U.S. Cl. 239/34; 239/36; 239/47; 239/55

(57) ABSTRACT

An air freshener has two hemispherical shells joined by a ring that includes a number of openings into the interior of the air freshener. A lip of the ring protrudes into the interior region and holds a scent element in place within the air freshener.

### BACKGROUND OF THE INVENTION

1. Field of the Invention

The present invention relates generally to the field of air fresheners. More specifically, the present invention discloses an air freshener in which the scent elements are held in place within the air freshener.

2. Statement of the Problem

A wide variety of air fresheners have been used for many years. For example, the present applicants have marketed spherical air fresheners under the SNEAKER BALLS® and SPEEDBALL® trademarks for many years. A cross-sectional view of this prior art air freshener is shown in FIG. 5. This air freshener includes two hemispherical plastic shells that are joined by a ring assembly with a number of openings that can be manually opened or closed to control the release of fragrance from within the air freshener. The fragrance is provided by an essential oil that gradually evaporates over time. This oil is carried by a porous paper ball that rolls freely within the air freshener. Earlier versions of these air fresheners employed a porous polyethylene disk that was also free to move about within the air freshener.

Both free-floating paper balls and disks have two drawbacks. First, the scent element can come to rest on the ring assembly and the essential oil sometimes leaks out of the air freshener through the openings in the ring assembly and stains the packaging or external objects. Second, neither a flat disk nor a paper ball offers a maximum amount of surface area. The surface area is important to maximize the release of the essential oil. Therefore, a need exists for an air freshener with a scent element that addresses these shortcomings.

3. Solution to the Problem.

The present invention addresses these concerns by employing scent elements that are held in place within the shell of the air freshener by the inner lip of the ring structure to help minimize the risk of leakage of essential oil. In addition, the scent elements deform during assembly of the air freshener to follow the generally hemispherical contour of the interior surfaces of the shells to thereby increase the surface area available for the release of essential oil.

### SUMMARY OF THE INVENTION

This invention provides an air freshener having two hemispherical shells joined by a ring that includes a number of openings into the interior of the air freshener. A lip of the ring protrudes into the interior region and holds a scent element in place within the air freshener.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is an exploded perspective view of the air freshener.

FIG. 2 is a cross-sectional view of the assembled air freshener.

FIG. 3 is a perspective view of the assembled air freshener.

FIG. 4a is a side elevational view of the air freshener with the ring assembly in the open position.

FIG. 4b is a side elevational view of the air freshener with the ring assembly in the closed position.

FIG. 5 is a cross-sectional view of a prior art air freshener.

FIG. 6 is a top view of a scent pad 30.

FIG. 7 is a side elevational view of a scent pad 30.

FIG. 8 is a top view of one of the rings 22, 24 in the ring assembly.

FIG. 9 is a side elevational view of one of the rings 22, 24 in the ring assembly.

FIG. 10 is a bottom view of one of the rings 22, 24 in the ring assembly.

### DETAILED DESCRIPTION OF THE INVENTION

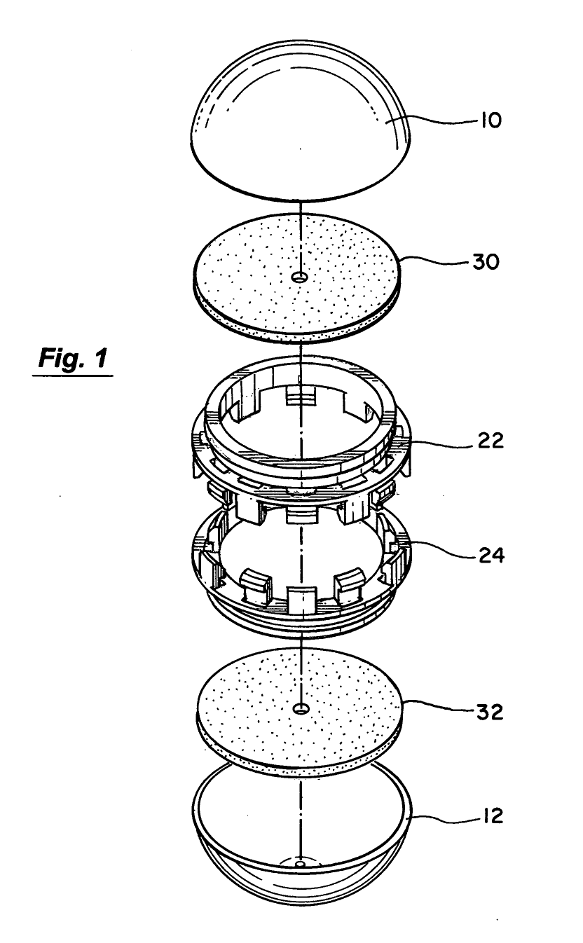

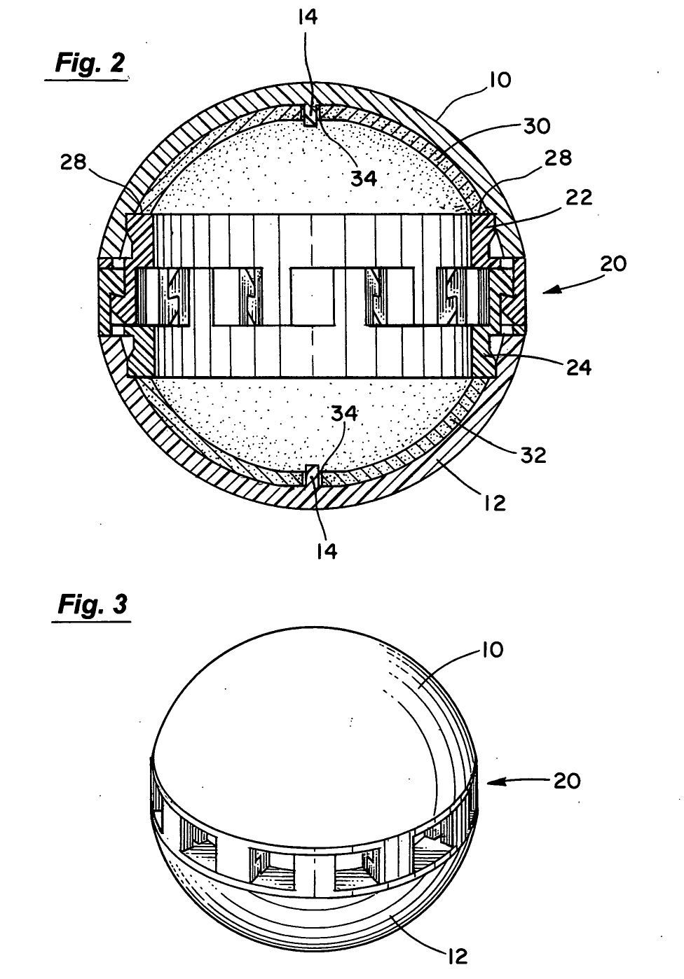

Turning to FIG. 1, an exploded perspective view is provided showing the major components of the air freshener. FIG. 2 is a corresponding cross-sectional view and FIG. 3 is a perspective view of the assembled air freshener. In this embodiment, two hemispherical shells 10 and 12 are joined together by a ring assembly 20 to form a generally spherical container to hold the scent elements 30 and 32.

In the preferred embodiment, both the upper and lower shells 10, 12 are made of molded plastic to produce a configuration with a generally hemispherical outer surface and a generally hemispherical inner surface. Both shells 10, 12 also have generally circular openings of similar diameters to facilitate joining the shells 10, 12 to the ring assembly 20. For example, the upper and lower shells 10 and 12 can be identical, which simplifies manufacture and reduces costs. However, the shapes and sizes of the shells 10, 12 are largely a matter of design choice and aesthetics.

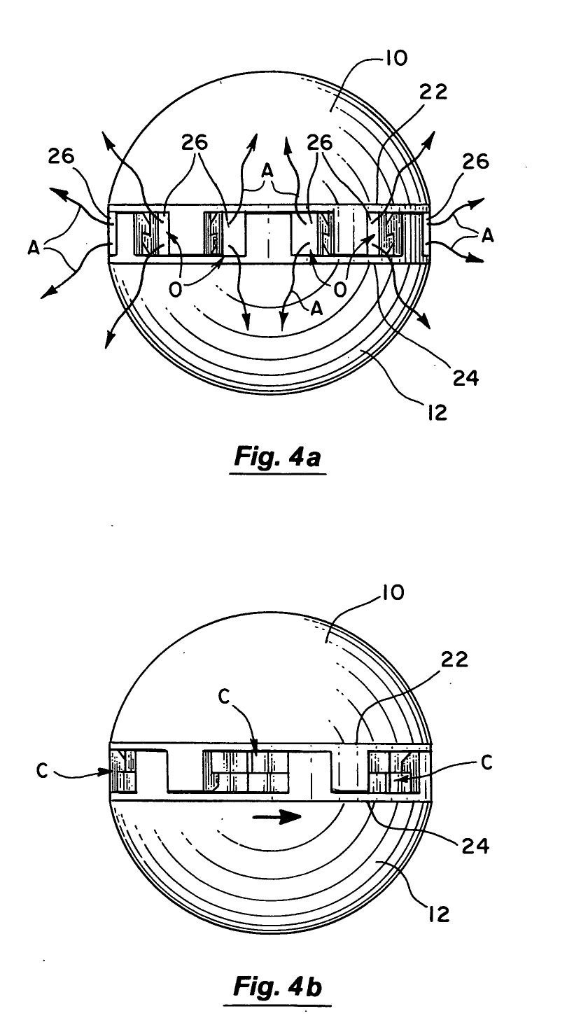

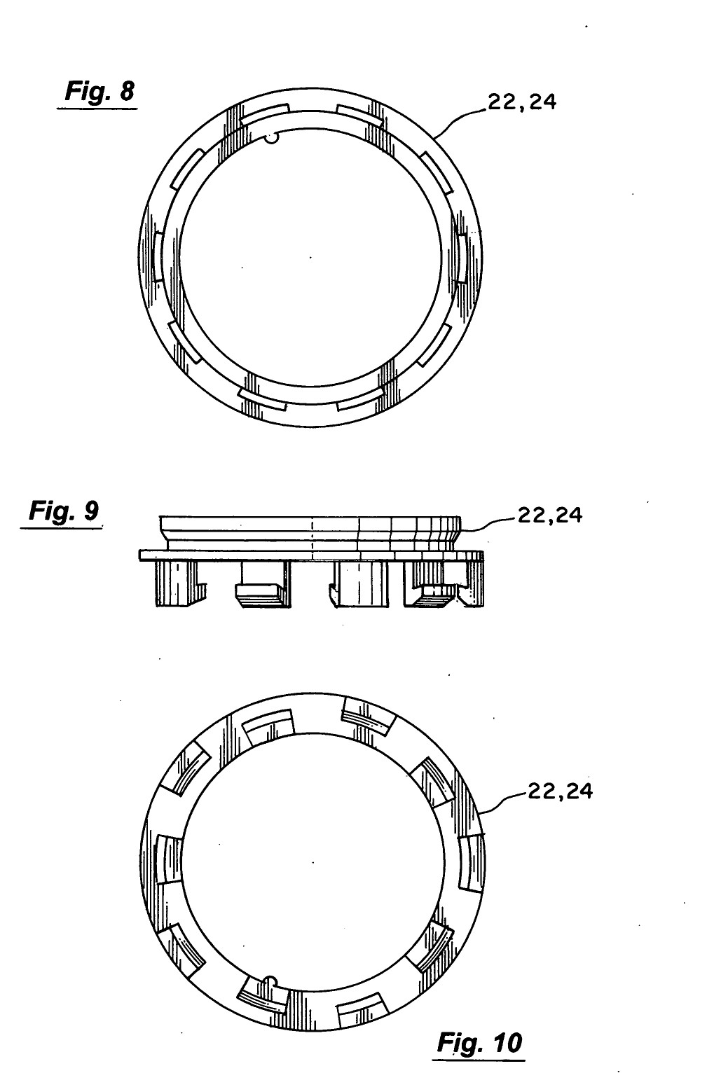

A ring assembly 20 is secured to the circular openings of the upper and lower shells 10, 12 by means of adhesive or the like to form a generally spherical container with an interior region to hold the scent elements 30, 32. A number of openings extend through the container to allow fragrance to escape from the scent elements 30, 32 in the surrounding atmosphere. In the embodiment shown in the drawings, the ring assembly 20 is made of an upper ring 22 and a lower ring 24 with interlocking teeth that engage one another, but allow a degree of rotation between the rings 22, 24. In the preferred embodiment, both rings 22 and 24 are identical to simplify manufacture and reduce costs. FIG. 8 is a top view, FIG. 9 is a side elevational view and FIG. 10 is a bottom view of an individual ring 22, 24. However, other configurations of the ring assembly could be readily substituted. As shown in FIGS. 4a and 4b, relative rotation of the rings 22, 24 allows the user to control the degree to which the openings 26 between the teeth of the rings 22, 24 are open or closed. For example, FIG. 4a shows the rings 22, 24 in the fully open position 0 to allow fragrance A to escape from within the air freshener. In contrast, FIG. 4b shows the rings 22, 24 in the fully closed position C. Optionally, openings could be formed through the walls of the shells 10, 12.

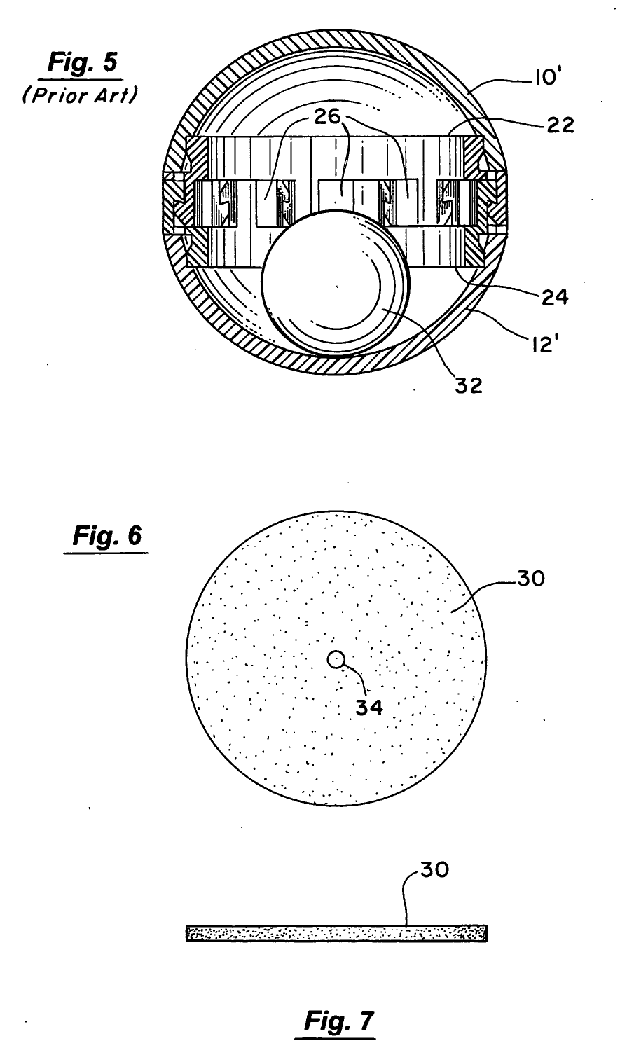

The scent elements 30 and 32 are held in place within the interior region of the air freshener by means of the lip 28 of the ring assembly 20 that protrudes into the interior region of the air freshener around at least a portion of the circular opening of the shells 10, 12, as shown in the cross-sectional view provided in FIG. 2. In the preferred embodiment, the scent elements 30, 32 are thin disks (e.g., 1⅜ inch diameter) with a circular peripheral rim. For example, the scent elements 30, 32 can be made of a non-woven material similar to a felt pad. FIG. 6 is a top view and FIG. 7 is a side elevational view of a scent pad 30 prior to insertion. The scent elements 30, 32 are thin enough to be forced up into each hemisphere of the air freshener and then locked in place by the lip 28 of the ring assembly 20 abutting the rim of the scent element 30, 32. The end result after insertion is that the scent elements 30, 32 are deformed into a curved or bowed shaped with one surface of the scent element abutting the interior surface of the shell 10, 12, as depicted in FIG. 2.

This approach allows two or more scent elements 30, 32 to be placed inside the air freshener (at least one in each hemispheric shell 10, 12) greatly increasing the amount of scent and the surface area for release. Thus, the present invention solves both of the problems associated with the prior art and is also easy to assemble. Because the scent pads 30, 32 are held in place against the interior surfaces of the shells 10, 12 and have only minimal contact with the ring assembly 20, essential oil is much less likely to leak out.

It should be noted that any number- of scent elements could be used, and that scent elements of a variety of shapes and dimensions could be used. In the specific embodiment shown in FIGS. 1 and 2, each scent element 30, 32, also includes a hole or indentation 34 that engages a small post or protrusion 14 on the interior surface of the shell 10, 12. These help to hold the scent elements 30, 32 in place within the air freshener.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of this invention as set forth in the following claims.

We claim:

1. An air freshener comprising:
a first shell with a circular opening;
a second shell with a circular opening;
a ring joining the circular openings of the shells to define an interior region within the shells, and having:
(a) a lip protruding into the interior region around at least a portion of the circular opening; and
(b) at least one opening into the interior region; and
a scent element held in place within the interior region by the lip of the ring.

2. The air freshener of claim 1 wherein at least one of the shells is generally hemispherical.

3. The air freshener of claim 1 wherein the scent element abuts the interior surface of one of the shells.

4. The air freshener of claim 1 wherein the joined shells and ring have a generally spherical shape.

5. The air freshener of claim 1 wherein the openings in the ring are closable.

6. The air freshener of claim 1 wherein the scent element comprises a pad.

7. The air freshener of claim 1 wherein the scent element comprises a disk having circular peripheral rim abutting the lip of the ring, and a surface abutting the interior surface of one of the shells.

8. An air freshener comprising:
a first shell with an interior surface and a circular opening;
a second shell with a circular opening;
a ring joining the circular openings of the shells to define an interior region within the shells, and having a lip protruding into the interior region around at least a portion of the circular opening;
at least one opening into the interior region; and
a generally circular scent element engaging the lip of the ring about its periphery and abutting the interior surface of the first shell.

9. The air freshener of claim 8 wherein at least one of the shells is generally hemispherical.

10. The air freshener of claim 8 wherein the joined shells and ring have a generally spherical shape.

11. The air freshener of claim 8 wherein the openings into the interior region pass through the ring.

12. The air freshener of claim 8 wherein the openings into the interior region are closable.

13. The air freshener of claim 8 wherein the scent element comprises a pad.

14. An air freshener comprising:
a first hemispherical shell with a hemispherical interior surface and a circular opening;
a second hemispherical shell with a hemispherical interior surface and a circular opening;
a ring joining the circular openings of the shells to define an interior region within the shells, and having:
(a) a lip protruding into the interior region around at least a portion of the circular opening; and
(b) at least one opening into the interior region;
a first generally-circular scent element engaging the lip of the ring about its periphery and abutting the interior surface of the first shell; and
a second generally-circular scent element engaging the lip of the ring about its periphery and abutting the interior surface of the second shell;
wherein the interior region between scent elements allows diffusion of scent from the scent elements through the openings in the ring.

15. The air freshener of claim 14 wherein the openings in the ring are closable.

16. The air freshener of claim 14 wherein the scent elements comprise pads.
